(19) United States
(12) Patent Application Publication
(10) Pub. No.: US 2011/0208236 A1
(43) Pub. Date: Aug. 25, 2011

(54) MUSCLE BIOPSY CLAMP

(76) Inventors: Samuel R. WARD, San Diego, CA (US); Richard L. LIEBER, Del Mar, CA (US)

(21) Appl. No.: 12/890,592

(22) Filed: Sep. 24, 2010

Related U.S. Application Data
(60) Provisional application No. 61/245,954, filed on Sep. 25, 2009.

(51) Int. Cl. A61B 17/28

(52) U.S. Cl. 606/205

(57) ABSTRACT

A muscle biopsy clamp device may be used to measure sarcomere lengths in vivo where laser diffraction may not be possible. The clamp device includes a jaw having serrated markings for securing a muscle bundle in the clamp device as well as to provide markings for the muscle biopsy as reference for the sarcomere length measurement.

### RELATED APPLICATIONS

This application claims the benefit of and priority to U.S. provisional patent application Ser. No. 61/245,954, filed 25 Sep. 2009, the contents of which is herein incorporated by reference in its entirety for any and all purposes.

### STATEMENT REGARDING FEDERALLY SPONSORED RESEARCH OR DEVELOPMENT

This invention was made with government support under HD048501 and HD050837 awarded by the NIH. The U.S. government may have certain rights in the invention.

### BACKGROUND OF THE INVENTION

Muscle architecture measurements provide quantitative estimates of muscle performance (Williams and Goldspink, 1978; Bodine et al., 1982; Powell et al., 1984). These values are critical input parameters for biomechanical modeling of the musculoskeletal system. However, architectural values only provide an estimate of the maximum force generating potential (Powell et al., 1984), maximum shortening velocity (Bodine et al., 1982), or maximum excursion (Williams and Goldspink, 1978) of a muscle. Muscle physiological properties, specifically the length-tension and force-velocity relations, provide a more functional understanding of muscle performance. In fact, these characteristics can modulate muscle force by 100%. Therefore, accurate estimates of a muscle's in vivo functional properties are required.

To characterize muscle sarcomere length-tension properties, investigators have traditionally relied on microscopy to estimate in vitro sarcomere length on a very small muscle sample. In the 1930's it was discovered that coherent light, when passed through a muscle cell, diffracted in such a way that sarcomere lengths could be measured (Sandow, 1936ab). Since that time, lasers have been used as the source of coherent light to measure sarcomere lengths in situ (Cleworth and Edman, 1972). This technique, which was fully developed in the 1980's and 1990's (Yeh et al., 1980; Lieber et al., 1984; Lieber and Boakes, 1988; Lieber et al., 1994), has been used to characterize the in vivo sarcomere length-joint angle relations of muscles in a variety of systems, including humans (Lieber and Boakes, 1988; Lieber and Brown, 1993; Lieber et al., 1994). The results of these studies have yielded valuable information to the modeling community regarding the in vivo length-tension behavior of muscle. However, in vivo laser diffraction can be a difficult, time consuming, and expensive endeavor that has only been performed in selected muscles. These muscles must be fairly superficial, exposed in a bloodless field, and have relatively simple architecture.

As can be seen, there is a need for understanding the in vivo sarcomere length-joint angle relations of many muscles that are anatomically deep and vulnerable to obscuring by blood as an alternative to laser diffraction. Thus, the present invention addresses shortcomings in the field of sarcomere length measurement by providing a novel muscle biopsy clamp and related system that allows sampling the sarcomere in situ and preserving sarcomere length.

### SUMMARY OF THE INVENTION

The present invention concerns a patentable muscle biopsy clamp device/system that, among numerous features, include various structural elements. Thus, one aspect of the invention relates to muscle biopsy clamps. The clamps of the invention comprise two securing arms that can be moved in relation to one another about a pivot, a jaw arm attached to each securing arm such that when the distal ends of the securing arms are moved apart the distal ends of the jaw arms also move apart to “open” the jaws, wherein the distal, jaw end of each jaw arm includes two or more jaw extensions that define a gap or opening between the jaw extensions. This arrangement is such that when a tissue, such muscle tissue, is clamped between the jaw arms of the clamp, the tissue is exposed between the jaw extensions.

A representative embodiment of this aspect of the invention is the clamp (10) shown in FIG. 1. The clamp shown has two securing arms (12) movably connected about a pivot, a jaw arm 14 attached to each securing arm (12) such that the distal end of the jaw arm defines a jaw (16). As those in the art will appreciate, such clamps can be opened and closed in order to secure a tissue sample, for example, bundles of muscle fiber in vivo, between the jaw extensions (shown in exemplary fashion as elements 16a and 16b in FIG. 5). In some preferred embodiments, the opposing surfaces of the jaw extensions (i.e., the tissue-contacting surfaces) can include structures, such as serrations, lands and grooves, etc. that allow for enhanced tissue securement, as compared to flat, smooth surfaces. The invention also includes embodiments wherein the opposing surfaces of the jaw extensions are coated with plastic, rubber, or other natural or synthetic material to enhance tissue grasping/securement and/or minimize damage to tissue clamped or secured between the jaws, particularly the jaw extensions.

In some preferred embodiments of this aspect, the jaw ends of the clamp can be detached from the jaw arms, which can be useful for storage and later analysis of tissue secured in the clamp's jaws. The invention also includes a receptacle adapted to receive detached jaw ends so that they can be stored, and, if desired, tissue clamped therein to be analyzed or stored.

In another aspect of the present invention, a muscle biopsy clamp comprises a muscle biopsy clamp according to the invention that includes one or more cutting blades for excising muscle (or other tissue) secured in the muscle biopsy clamp device.

Another aspect of the invention concerns tissue biopsy methods. Such methods involve securing a tissue, for example, muscle tissue, sample in the jaws of a muscle biopsy clamp according to the invention and excising the tissue sample secured in the jaws of the clamp.

Still another aspect of the invention concerns methods for measuring muscle sacromere length. Such methods comprise securing a muscle tissue sample in the jaws of a muscle biopsy clamp according to claim 1 and measuring the length of the secured muscle sacromeres.

These and other features, aspects and advantages of the present invention will become better understood with reference to the following drawings, detailed description, and claims.

### BRIEF DESCRIPTION OF THE DRAWINGS

FIG. 1 is a side view of a muscle biopsy clamp device according to an embodiment of the present invention;

FIG. 2 is a top view of the muscle biopsy clamp device of FIG. 1;

FIG. 3 is an end view of the muscle biopsy clamp device of FIG. 1;

FIG. 4 is a perspective view of the muscle biopsy clamp device of FIG. 1;

FIG. 5 is a close-up view of the region A of FIG. 4;

FIG. 6 is side view of a muscle biopsy clamp device according to an embodiment of the present invention, including a close-up end view thereof;

FIGS. 7A and 7B show pictures of sarcomere length measurements on a tibialis anterior muscle fascicle, wherein FIG. 7A shows a laser diffraction device beneath fascicle to measure sarcomere length, and FIG. 7B shows the muscle biopsy clamp, according to an embodiment of the present invention, in place to harvest the fascicle;

FIG. 8A shows a scatter plot of diffraction-based sarcomere lengths (x-axis) vs. clamp-based sarcomere lengths (y-axis) demonstrating excellent agreement between the two techniques;

FIG. 8B shows a scatter plot of the magnitude of clamp-based error (y-axis) vs. diffraction-based sarcomere length (x-axis), noting that acceptable error (˜10%) can be obtained above 2.6 μm; and

FIG. 8C shows sarcomere length data obtained in the clamp before and after fixation (y-axis), demonstrating that fixation is not the primary source of error in the method described herein.

### DETAILED DESCRIPTION OF THE INVENTION

The following detailed description is not to be taken in a limiting sense, but is made merely for the purpose of illustrating the general principles of the invention, supporting the appended claims.

Various features of the invention, as described below, are capable of being used independently of one another or, alternatively, in combination with one or another features of said invention.

Broadly, embodiments of the present invention provide for devices useful for measuring in vivo sarcomere lengths in muscle tissues where measurement methodologies such as laser diffraction, for example, may not be possible.

In a first preferred embodiment of the present invention provides a muscle biopsy clamp that can be used to quickly and accurately take in vivo samples of muscle so that sarcomere length can be assessed and/or other analyses of the tissue performed, either in vivo or after the tissue secured in the clamp has been excides, if desired.

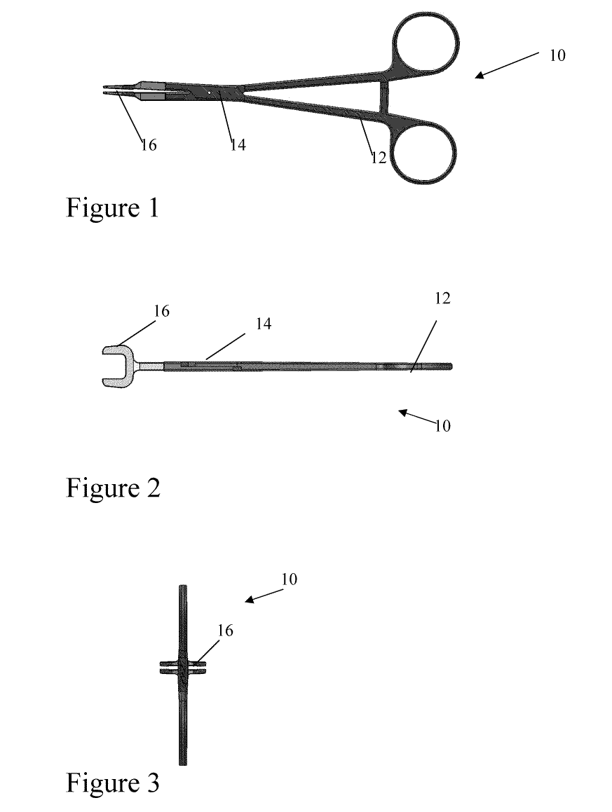

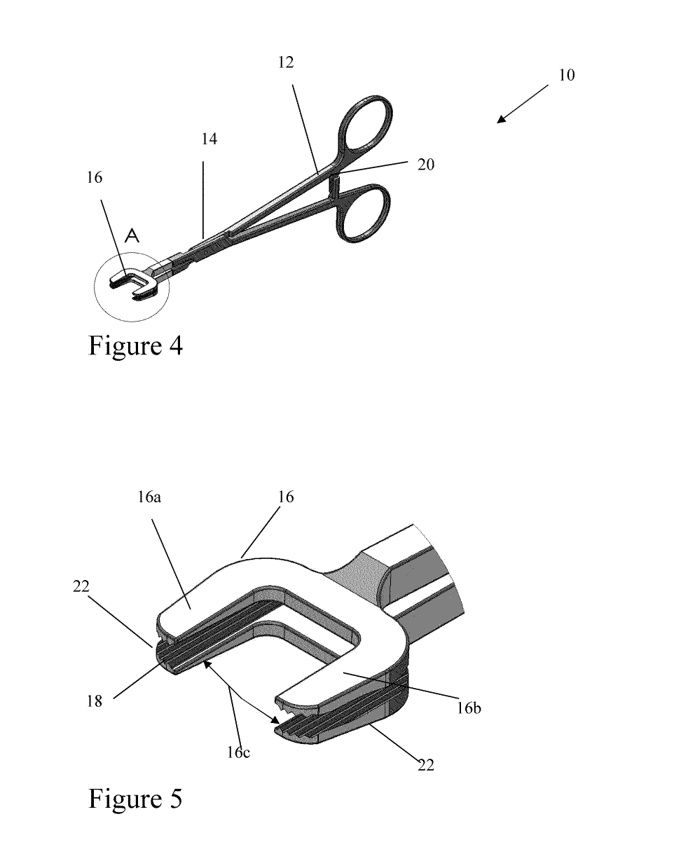

Referring to FIGS. 1 through 5, various views of a muscle biopsy clamp device 10 are shown. Briefly, each clamp at least comprises a pair of securing arms (12) that can be moved in relation to each other about a pivot, joint, or similar mechanical joining structure, a jaw arm (14) attached to each securing arm, and a jaw (16) disposed at the end of each jaw arm (14) opposite the securing arm (12). Such clamps also preferably include a structure such as a finger loop or ring or other structure at the distal end of one or both securing arms (12) opposite the jaw arms (14) so as to allow a user to securely hold or control the clamp. Also, the clamps of the invention also preferably include a structure that allows the clamp to be reversibly secured in clamped, or closed position, preferably to deliver a desired amount of clamping pressure between the jaws (16). Such structures include toothed ratchet bars attached to each securing arm (12) that are designed to matingly engage when the securing arms (12) are squeezed for closure. The mating engagement of the ratchet bars can be reversed or released by a user, if an when desired.

The clamp shown in FIG. 1 comprises two opposing lever-acting structures, each a securing arm (12), connected to each other at one end about a fulcrum or pivot point, wherein the portion of the structure or component past the pivot point is termed herein a jaw arm (14). In some embodiments, a clamp according to the invention can be adapted from a conventional hemostat design such that force applied to the securing arms (12) to close the space between them results in the jaw arms (14) closing together and the attached jaws (16) clamping shut. In some of these embodiments, the device is hand-held and manually operated to cause clamping of the jaws to a desired pressure by ratchet arms (20). Alternatively, a clamp of the invention can employ any suitable mechanical or automated device to control the closing and/or opening pressure applied to the jaws via the securing arms.

The clamps of the invention are typically made from metals, plastics, or composite materials suitable for the intended application. Whichever material is selected, it can be formed or shaped using any suitable process, for example, casting, extrusion, forging, injection molding, etc.

The jaw (16) can include surface contours or structures such serrations 18 (see, in particular, FIG. 5), lands and grooves, ribs, posts, etc. on a portion thereof, typically on one or more of the surfaces of the jaw extensions (or additional structures, such as covers, that may optionally be attached thereto) intended to contact the tissue to be clamped. The serrated markings 18 (or comparable structures) aid the secure immobilization of the tissue, for example, a muscle bundle (not shown), in the in situ position. Serrations and other structures can also serve as landmarks for making measurements. Similarly, some such structures can also produce marks and the like on the clamped tissue that can visualized and, if desired, used, for example, to provide markings for the muscle biopsy as reference for the sarcomere length measurements.

A jaw 16 can include a first and second jaw extension 16a, 16b separated by a jaw opening or spacing 16c. Jaw extensions can be formed in any desired shape or configuration to provide the desired jaw opening. As already described, the portions of a jaw intended for contacting tissue upon clamping (e.g., jaw extensions) can be covered with materials intended to facilitate tissue securement upon clamping, reduce tissue damage, etc. Such materials include natural and synthetic materials. If desired, covers can be disposable.

In any event, after tissue such as muscle is secured between the jaws 16 by supplying the desired force to the securing arms (either manually or through the use of another manually operated or automated device), in preferred embodiments the clamp device 10 can be secured by a structure such as interlocking ratchet arms 20. If a biopsy is desired, the muscle specimen clamped between jaws 16 may be excised. This can be accomplished in any suitable way. In some embodiments, biopsy excision is accomplished by using a cutting tool such as scalpel. Alternatively, however, a clamp according to the invention can be configured to accomplish excision of tissue clamped in the jaws. In some of these embodiments, the clamp device of invention has affixed along the outer side edges of the jaw extensions 16a and 16b one or more cutting blades 22 disposed such that when the jaws are being clamped down on the specimen, the cutting blades will snip through the clamped tissue (e.g., muscle) to obtain the biopsy. The biopsy can then, for example, be directly used for measurement or analysis, deposited into a fixation solution, etc. As will be appreciated, a suitable receptacle adapted to receive the jaws, particularly jaws that can be removed from the clamp, can be used to receive the jaws and the tissue clamped therein, be it for analysis, fixation, freezing, etc.

Thus, in still further embodiments, the clamp includes jaws that can be removed from the jaw arms. Typically, using such embodiments, the jaws will be removed together in the clamped position after a tissue sample has been clamped therein so that the sample can be retained as muscle biopsy sample and, if desire, fixed between the clamped jaw extensions. In such embodiments, the jaw arms and jaws are detachable, and will include suitable structures that allow each jaw to be readily removed from the distal end of its corresponding jaw arm.

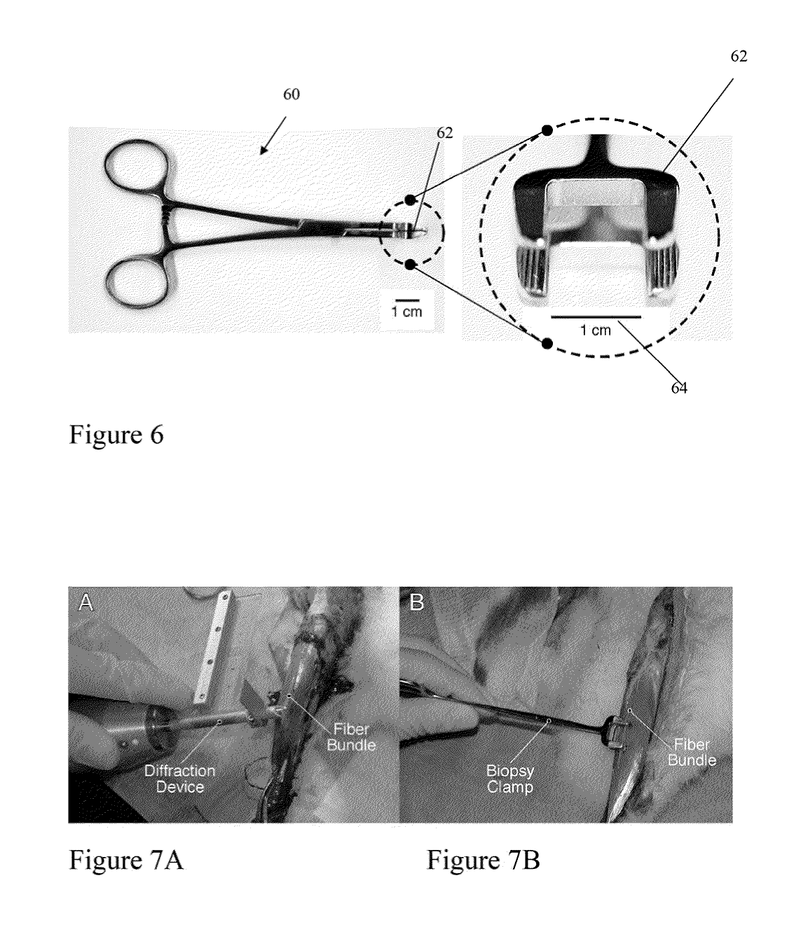

Referring now to FIG. 6, there is shown a muscle biopsy clamp device 60 having a jaw 62 with a jaw opening 64 of 1 centimeter (cm). It should be noted that the jaw opening of 1 cm is exemplary of various jaw opening widths useful for sampling, such widths ranging from about 0.1 cm or less up to about 10 cm, preferably from about 0.5 cm to about 2 cm, more preferably from about 0.5 cm to 1.5 cm, which are useful in certain embodiments of the present invention, depending, for example, on the tissue type to be studied or biopsied.

In the context of the invention, a securing arm can be any suitable length, although lengths ranging from about 5 cm to about 30 cm in length are preferred. Several exemplary securing arm lengths include 5 cm, 15 cm, and 30 cm. Preferably, a securing arm is longer, typically much longer, than the jaw arm attached thereto. For clarity, herein it will be understood that jaw arms and securing arms can be separately formed components that are then securely attached such that force applied to the securing arm can be effectively transferred to the jaw arm and jaw. Alternatively, and preferably, the securing and jaw arms are actually part of the same component, and are formed together. In any event, a corresponding jaw arm and securing arm will be understood to function as a single unit, with the different regions, i.e., the “securing arm” and “jaw arm” being conceptually referenced for purposes of understanding and describing the invention and not to describe separate, physically distinct components unless specified. In some embodiments, a securing arm is 2-10 or more times the length of the corresponding jaw arm. That said, preferred jaw arm lengths range from about 3 cm to about 7 cm in length. Several exemplary jaw arm lengths include 3 cm, 5 cm, and 7 cm. As those in the art will understand, the selection of the particular lengths of the securing arm and jaw arm for a particular clamp design will depend on factors such as the intended application, the tissue to be clamped, whether the jaws can be detached from the jaw arms, etc. For example, axis spring tension may be matched from a particular combination of securing arm length and jaw arm length.

As already described, in some embodiments the jaws of the clamp of the invention are designed to be separated or detached from the jaw arm to which they are connected. Any suitable attachment/detachment mechanism can be employed, resulting in a jaw that can detached from corresponding jaw arm. In some of these embodiments, the jaws can include one or more cutting element or blades so that after the clamp's jaws are closed and the muscle bundle secured, the cutting blade(s) can excise the muscle sample from the muscle bundle. For clarity, herein it will be understood that jaw arms, jaws, and jaw extensions can be separately formed components that are then securely attached to other components. Alternatively, jaw arms, jaws, and jaw extensions are part of the same component, and are formed together. In any event, a jaw arm, jaw, or jaw extensions will be understood to function as a single unit, with the different regions, i.e., the “jaw arm”, “jaw”, or “jaw extensions” being conceptually referenced for purposes of understanding and describing the invention and not to describe separate, physically distinct components unless specified.

The user performs detachment of the jaws from the jaw arms. In preferred embodiments, the clamped jaws remain clamped, or locked, upon removal from the device so that the tissue secured therein remains securely clamped after the jaws have been disconnected or otherwise detached from the jaw arms of the clamp. Any suitable locking mechanism can be employed. As will be appreciated, after detachment of the jaws, they may be disposed of, in which event another paid of complementary jaws can be securely attached to the jaw arms. The invention envisions clamps having both disposable, single use jaws or jaws that can be attached and detached repeatedly from their corresponding jaw arms.

As already described, in some embodiments the muscle biopsy clamp device of the invention is part of a muscle biopsy system. Typically, such systems include a clamp according to the invention, particularly clamps configured to have locking, detachable jaws, and a receptacle adapted to receive the jaws. In preferred embodiments, such receptacles are made from materials that suitable for fixation, analysis, and/or storage of the tissue sample clamped in the jaws.

Another aspect of the invention concerns uses of the clamps of the invention. One particularly preferred use is the measurement of in vivo muscle sarcomere length. The measurement of in vivo muscle sarcomere length facilitates the definition of in vivo muscle functional properties and comparison of muscle design amongst functional muscle groups. In vivo sarcomere lengths are available for just a handful of human muscles, largely due to the technical challenges associated with their measurement.

The application of the invention is described in following Examples. The scope of the invention is not to be considered limited thereto.

### EXAMPLE I

Fourteen centimeter long stainless steel hemostat clamps (Model 3-113-14, Sabri Group, Pompano Beach, Fla., USA) were modified by attaching custom 1 cm wide serrated jaws to their ends (See FIGS. 1 through 6). The jaws were machined from stainless steel (316) blocks using wire electrical discharge machining (EDM) to achieve very tight tolerances (±0.0005 cm) between mating jaw serrations (See FIGS. 5 and 6). After the jaws were machined, they were welded to the hemostat jaws using tungsten inert gas (TIG) and polished. This machining process allowed the jaws to be sterilized using standard autoclaving and provided sufficient clamping pressure to prevent slippage of muscle fibers between the jaws.

To validate sarcomere lengths obtained using the clamp-based method, biopsies (n=23) of the tibialis anterior muscles of New Zealand White rabbits (n=19) were sampled. Animals were induced and maintained under gas anesthesia (isoflurane 2%) in accordance with the Veterans Administration Institutional Animal Care and Use Committee's approval.

The skin and fascia covering the anterior compartment was incised and reflected to expose the muscle. Micro-Adson forceps (Model 11018-12, Fine Science Tools, Foster City, Calif., USA) and Metzenbaum scissors (Model 14018-13, Fine Science Tools, Foster City, Calif., USA) were used to isolate small tibialis anterior fiber bundles approximately 2 cm in length. An intraoperative laser diffraction device (Lieber et al. 1994) was then placed deep to the isolated bundle, taking care to maintain the in situ trajectory of the muscle fibers (FIG. 7A). The foot was then placed in a position between full dorsiflexion and maximum plantar flexion to capture the full range of passive sarcomere lengths available in the tibialis anterior muscle. Once a foot position was chosen, the laser was inserted beneath the bundle and the distance between the +1 to −1 or +2 to −2 diffraction bands was measured and converted to sarcomere length as previously described (Lieber et al., 1994). This value was used as the “gold standard” sarcomere length value as it has been shown to represent sarcomere lengths throughout a passive muscle (Takahashi et al., 2007). Maintaining the foot position, the laser tip was then removed from the muscle and the biopsy clamp was placed around the fiber bundle in the same location as the laser tip. The jaws were closed (FIG. 7B) and the fiber bundle was cut proximally and distally to the clamp, removed with the muscle fibers, and submerged in Formalin. In some cases (n=4) two biopsies were taken from the same muscles at different joint positions. After 24 hours of fixation, the fiber bundle was removed from the assembly and placed on a glass slide for a second laser diffraction measurement (Lieber and Blevins, 1989).

In a subset of biopsies (n=16), sarcomere lengths were measured in situ, after clamping alone, and after clamping with subsequent fixation. This allowed us to determine if the source of the sarcomere length measurement error was related to the fixation process or the clamping process.

The intraclass correlation coefficient (equation_{2,1}), simple linear regression, and average percent error was used to validate the technique. Data are presented as mean±SE, α was set at 0.05, and data were analyzed using SPSS (version 16.0, SPSS, Chicago, Ill.).

### RESULTS AND DISCUSSION

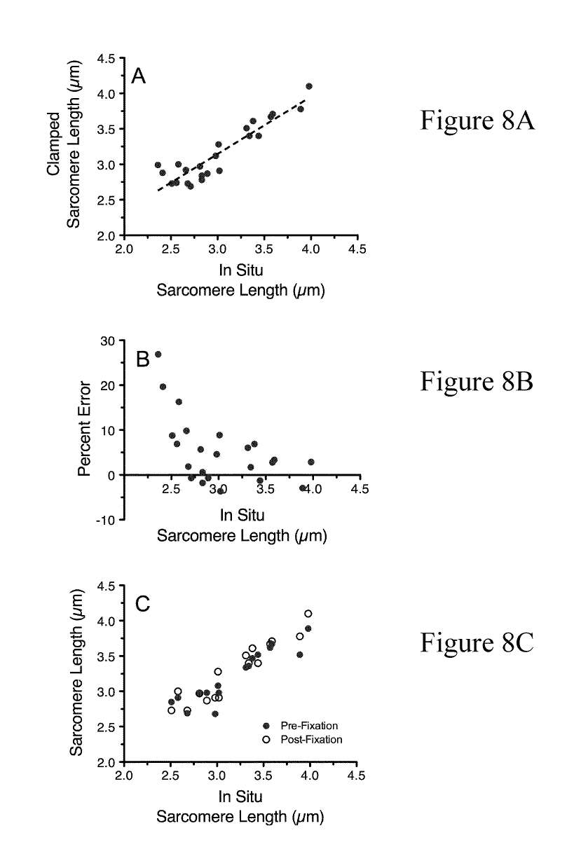

Analysis of data from all 23 biopsies revealed that there was excellent agreement (ICC_{2,1}=0.929, r²=0.77, average error=5.3%) between diffraction-based and biopsy-based sarcomere lengths (FIG. 8A). However, it was apparent that biopsy-based sarcomere length error was larger when in vivo sarcomere length was less than 2.6 μm (FIG. 8B). When sarcomere lengths were restricted to values greater than 2.6 μm, the agreement between techniques was even better (ICC_{2,1}=0.972, r²=0.92, average error=2.5%). This is an interesting finding in light of the fact that rabbit muscle fibers would be expected to generate significant passive tension at lengths greater than 2.6 μm. This suggests that the biopsy clamp may apply a slight longitudinal tension to the muscle fibers during the clamping process when they are on slack. However, when fibers are under moderate passive tension (i.e. at sarcomere lengths greater than 2.6 μm), the fibers resist the small longitudinal tension created by clamping which allows the clamp-based method to yield more accurate sarcomere lengths.

To characterize the source of the clamp-based measurement error, some biopsies were measured in situ, immediately after clamping, and after clamping and fixation. These data demonstrated that fixation did not introduce systematic sarcomere length error (FIG. 8C); rather, the clamping process at short sarcomere lengths introduced the error (FIG. 8C).

In vivo sarcomere length can be estimated accurately using a muscle biopsy clamp that preserves in vivo sarcomere lengths. Using a simple goniometer, joint angle can be accurately measured (Gogia et al., 1987), providing a reference position for sarcomere length measurements. Together, these tools will allow sarcomere length-joint angle relationships to be defined for a greater variety of muscles (i.e. non superficial) than could be studied by laser diffraction. Although the described technique still used laser diffraction to obtain sarcomere length of fixed tissue, light microscopy could also easily be used to measure sarcomere lengths after fixation (Huxley and Peachy, 1961).

When using these data as input variables for musculoskeletal models it is important to consider that the biopsy allows average sarcomere length to be measured in a single bundle of muscle fibers. Although it has been demonstrated that a single sarcomere length value represents whole muscle average sarcomere lengths in passive muscle (Takahashi et al., 2007), it has been suggested that sarcomere lengths may vary regionally within active muscles due to extramuscular myofascial force transmission (Maas et al., 2003; Yucesoy et al., 2006; Yucesoy et al., 2007), heterogeneous tendon strains (Lieber et al., 1992), or regional moment arm differences in muscles with broad insertions (Blemker and Delp, 2005; Blemker et al., 2005). Additionally, it has been suggested (Herzog and ter Keurs, 1988) that sarcomere length—tension relations do not precisely scale to whole muscle length—tension relations in all cases. Therefore, biopsy-based sarcomere length—joint angle data only yield an estimate of whole muscle length—tension behavior. Accurate modeled muscle forces may require additional information to achieve the level of accuracy required for an individual experiment.

In the development stage of the clamp according to an embodiment of the present invention, jaw widths of 0.5 cm, 1.0 cm, and 1.5 cm were tested. In terms of EDM fabrication, although jaws of 0.5 cm and 1.5 cm can be used, in a preferred embodiment a jaw width of 1.0 cm provides a large enough sample to obtain accurate sarcomere lengths and harvest a relatively small length of a muscle fascicle.

There are several technical advantages to this technique over laser diffraction. First, laser diffraction is vulnerable to light scattering by blood and connective tissue in the surgical field. In these cases, a visible diffraction pattern simply cannot be obtained. Second, laser diffraction is limited to relatively shallow and wide surgical exposures. In cases where the muscle of interest is deep (e.g. spinal musculature and hip musculature) or the surgical field is narrow (e.g. minimally invasive surgical techniques), the laser tip cannot be placed deep to a muscle fiber bundle without elevating the bundle, which yields artificially long sarcomere lengths. Third, hand-held intraoperative lasers are expensive and require special sterilization (i.e. gas or gas-plasma) to preserve the electronic circuitry. Finally, intraoperative laser diffraction requires significant practice to achieve the skill level required to obtain accurate measurements in the very narrow time window provided during surgery.

Given the intraoperative speed and simplicity of the techniques described herein and the relatively low-cost of a biopsy clamp according to the invention, this invention will find application is many areas, including the taking of biopsies.

### REFERENCES

The below references, described above by author and date, are herein incorporated by reference:
Blemker, S. S., Delp, S. L., 2005, Ann Biomed Eng 33(5), 661-73.
Blemker, S. S., Pinsky, P. M., Delp, S. L., 2005, J Biomech 38(4), 657-65.
Bodine, S. C., Roy, R. R., Meadows, D. A., Zernicke, R. F., Sacks, R. D., Fournier, M., Edgerton, V. R., 1982, Journal of Neurophysiology 48, 192-201.
Cleworth, D. R., Edman, K. A. P., 1972, Journal of Physiology 227, 1.
Gogia, P. P., Braatz, J. H., Rose, S. J., Norton, B. J., 1987, Phys Ther 67(2), 192-5.
Herzog, W., ter Keurs, H. E., 1988, Pflugers Arch 411(6), 642-7.
Huxley, A. F., Peachy, L. D., 1961, Journal of Physiology (London) 156, 150-165.
Lieber, R. L., Blevins, F. T., 1989, Journal of Morphology 199, 93-101.
Lieber, R. L., Boakes, J. L., 1988, American Journal of Physiology 254, C759-C768.
Lieber, R. L., Brown, C. G., 1993, A
Lieber, R. L., Brown, C. G., Trestik, C. L., 1992, Journal of Miomechanics 25, 421.
Lieber, R. L., Loren, G. J., Fridén, J., 1994, Journal of Neurophysiology 71, 874-881.
Lieber, R. L., Yeh, Y., Baskin, R. J., 1984, Biophysical Journal 45, 1007-1016.
Maas, H., Baan, G. C., Huijing, P. A., Yucesoy, C. A., Koopman, B. H., Grootenboer, H. J., 2003, J Biomech Eng 125(5), 745-53.
Powell, P. L., Roy, R. R., Kanim, P., Bello, M., Edgerton, V. R., 1984, Journal of Applied Physiology 57, 1715-1721.
Sandow, A., 1936a, Journal of Cell Comparative Physiology 9, 55-75.
Sandow, A., 1936b, Journal of Cell Comparative Physiology 9, 37-54.
Takahashi, M., Ward, S. R., Lieber, R. L., 2007, J Hand Surg [Am] 32(5), 612-7.
Williams, P., Goldspink, G., 1978, Journal of Anatomy 127, 459-468.
Yeh, Y., Baskin, R. J., Lieber, R. L., Roos, K. P., 1980, Biophysical Journal 29, 509-522.
Yucesoy, C. A., Koopman, B. H., Grootenboer, H. J., Huijing, P. A., 2007, Biomech Model Mechanobiol 6(4), 227-43.
Yucesoy, C. A., Maas, H., Koopman, B. H., Grootenboer, H. J., Huijing, P. A., 2006, Med Eng Phys 28(3), 214-26.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

It should be understood, of course, that the foregoing relates to exemplary embodiments of the invention and that modifications may be made without departing from the spirit and scope of the invention as set forth in the following claims. The terms and expressions which have been employed are used as terms of description and not of limitation, and there is no intention that use of such terms and expressions imply excluding any equivalents of the features shown and described in whole or in part thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A muscle biopsy clamp, comprising a first securing arm attached to a first jaw arm pivotally connected to a second securing arm attached to a second jaw arm, wherein the first jaw arm has a first jaw comprised of two jaw extensions that define a first jaw opening and the second jaw arm has a second jaw comprised of two jaw extensions that define a second jaw opening.

2. A muscle biopsy clamp according to claim 1 wherein the jaw extensions of the first and second jaws comprise a serrated, grooved, ribbed, or textured tissue-contacting surface.

3. A muscle biopsy clamp according to claim 1 wherein the first and second jaws are detachable from the first and second jaw arms.

4. A muscle biopsy clamp according to claim 1 wherein the first and second securing arms each further comprise a toothed ratchet bar configured to engage a complementary toothed ratchet bar on the other securing arm.

5. A muscle biopsy clamp according to claim 1 wherein the first and second jaw openings each independently range from about 0.1 cm to about 10 cm, optionally from about 0.5 cm to about 1.5 cm.

6. A muscle biopsy clamp according to claim 1 wherein the first and second jaws comprise cutting elements to excise tissue secured in the jaws.

7. A muscle biopsy system, comprising a muscle biopsy clamp according to claim 1 and a receptacle configured to receive the jaws from the muscle biopsy clamp.

8. A muscle biopsy system according to claim 7 wherein the first and second jaws comprise cutting elements to excise tissue secured in the jaws.

9. A method for measuring muscle sacromere length, comprising securing a muscle tissue sample in the jaws of a muscle biopsy clamp according to claim 1 and measuring the length of the secured muscle sacromeres.

10. A tissue biopsy method, comprising securing a tissue sample in the jaws of a muscle biopsy clamp according to claim 1 and excising the tissue sample secured in the jaws of the muscle biopsy clamp.
